(19) United States
(12) Patent Application Publication
(10) Pub. No.: US 2011/0295299 A1
(43) Pub. Date: Dec. 1, 2011

(54) GASTRIC VOLUME FILLING CONSTRUCT

(76) Inventors: Gavin Braithwaite, Cambridge, MA (US); Martin van Buren, Chelmsford, MA (US); Stephen Spiegelberg, Winchester, MA (US); Shantanu Gaur, Canonsburg, PA (US); Samuel Levy, Columbus, OH (US); Jonathan Wecker, Weston, MA (US); Ram Chuttani, Dover, MA (US)

(21) Appl. No.: 13/039,067

(22) Filed: Mar. 2, 2011

Related U.S. Application Data
(60) Provisional application No. 61/310,190, filed on Mar. 3, 2010.
(60) Provisional application No. 61/437,233, filed on Jan. 28, 2011.

(51) Int. Cl. A61M 29/04

(52) U.S. Cl. 606/191

(57) ABSTRACT

The technology described herein provides methods for addressing obesity by introducing a device into the stomach. Embodiments of a device for treating obesity may comprise a shaped membrane construct that contains a swellable material. The construct may be contained within a capsule which is ingested by a patient. When the capsule dissolves in the stomach, the gastric secretions may diffuse through the membrane and spontaneously swell the internal material, which swells and stiffens the construct sufficiently to create a sensation of satiety by both filling gastric volume and by distending the walls of the stomach. The volume of the construct may be sufficient to prevent passage through the pyloric sphincter. After a set period of time or after the administration of a degradation formulation, the device may structurally degrade to allow passage through the pyloric sphincter and eventual passage from the body.

### RELATED APPLICATIONS

This application claims priority under 35 U.S.C. §119(e) to U.S. Provisional Application Ser. No. 61/310,190, titled “SYSTEM AND METHOD FOR DEPLOYMENT OF INTRAGASTRIC CONSTRUCT,” filed on Mar. 3, 2010, and to U.S. Provisional Application Ser. No. 61/437,233, titled “SYSTEM AND METHOD FOR DEPLOYMENT OF INTRAGASTRIC CONSTRUCT FOR WEIGHT LOSS WITH ON-DEMAND DEGRADATION,” filed on Jan. 28, 2011, both of which are herein incorporated by reference in their entirety.

### BACKGROUND

The National Institutes of Health reports that in 2009, an estimated 133 6 million Americans were either overweight or obese. This figure represents over 40% of the U.S. population, a dramatic increase from 25% as recently as 1980. It is anticipated that the advances made through national smoking cessation efforts will be entirely negated by the adverse and widespread health effects of the American obesity epidemic, which include diabetes, coronary artery disease and hypertension. Aspects and embodiments of the present invention generally relate to the field of medical devices and methods for promoting weight loss, and more particularly promoting weight loss through non-pharmaceutical appetite suppression.

Typical weight loss treatment modalities, excluding diet and exercise, have focused either on promoting malabsorption (for example, pharmaceuticals and/or endoluminal sleeves) or restricting gastric volume (for example, gastric bypass surgery, gastric bands, and/or intragastric balloons). Currently available pharmaceutical treatments have yet to gain traction in light of their toxicity, side effect profiles and transient effectiveness. Generally, available surgical interventions remain highly invasive, expensive, and only applicable to morbidly obese patients.

Methods of procedurally-assisted weight loss approved for use outside of the United States include placement of an intragastric balloon. These balloons, which are placed and later removed with an endoscope, are usually composed of silicone and are inflated once placed in the stomach. Intragastric balloons have been shown to be highly effective in inducing weight loss during a typical six month gastric residence; however, patient discomfort is common, and the long-term placement of these devices can cause ulceration of the gastric mucosa and small bowel obstruction secondary to spontaneous deflation. Swallowable, gas-fired intragastric balloons have also been previously described.

Expandable polymer systems comprised of hydrogels or cellulose derivatives have been described previously but typically suffer from limited efficacy and numerous disadvantages. Residence time in the stomach is typically less than six hours, requiring a substantial and rigorous regimen of treatment compliance to provide round-the-clock satiety. Insufficient structural integrity in the inhospitable gastric milieu typically drives this short residence time. Gastric peristalsis exerts considerable force on these formulations resulting in fragments small enough to pass through the pyloric outlet.

### SUMMARY

Aspects and embodiments of the present invention relate to apparatus, systems, and methods for encouraging weight loss through appetite suppression. In some embodiments, a system comprises a gastric construct, an apparatus to deliver the construct to the stomach, and a degradation formulation. Aspects and embodiments of the present invention further relate to a method of fabricating a gastric construct.

In accordance with an aspect of the present application, there is provided a construct for inducing weight loss in a patient. The construct comprises an expandable device constructed and arranged to enter a stomach of a patient in a first compressed state, and responsive to insertion into the stomach, to expand into a second expanded state. A portion of the expandable device is constructed to structurally degrade responsive to contact with a high pH fluid. In accordance with some embodiments, the second expanded state comprises an open skeletal structure.

In accordance with some embodiments, the expandable device includes one or more fluid-permeable membrane panels defining at least part of an inner volume and a filling material contained in the inner volume, the filling material including a substance which absorbs moisture and swells in the presence of gastric fluids. In accordance with some embodiments, the one or more fluid-permeable membrane panels and the filling material are constructed and arranged such that swelling of the filling material causes the expandable device to expand into the second expanded state. In accordance with some embodiments, the expandable device is constructed and arranged such that in the presence of a high pH fluid an opening forms into the inner volume, the opening having a size sufficient to allow the filling material to escape the inner volume.

In accordance with some embodiments, the expandable device is structurally stable in a gastric environment with a pH of less than about 7. In accordance with some embodiments, the portion of the expandable device structurally degrades in a gastric environment with a pH greater than about 7. In accordance with some embodiments, the portion of the expandable device structurally degrades in a gastric environment with a pH greater than about 8.

In accordance with some embodiments, the one or more fluid-permeable membrane panels comprise one of polyester, poly-tetra-fluoro-ethylene, and nylon.

In accordance with some embodiments, the expandable device further includes a joining material coupled to one or more fluid-permeable membrane panels and constructed to join a first to portion of the one or more fluid-permeable membrane panels to one of a second portion of the one or more fluid-permeable membrane panels and a second membrane panel to form the inner volume. The joining material may be structurally stable in a gastric environment with a pH of less than about 7. The joining material may degrade in a gastric environment with a pH greater than about 7. The joining material may degrade in a gastric environment with a pH greater than about 8. In accordance with some embodiments, the joining material comprises stitching including a filament formed of one of poly(methacrylic acid-co-methyl methacrylate), poly(methyl acrylate-co-methyl methacrylate-co-methacrylic acid) co-polymer, and another methacrylic acid-methyl methacrylate co-polymer.

In accordance with some embodiments, the one or more fluid-permeable membrane panels comprise a plurality of fluid-permeable membrane panels coupled together using the joining material. The one or more fluid-permeable membrane panels may each have a maximum dimension of less than about 10 cm.

In accordance with some embodiments, the construct further comprises a dissolvable capsule containing the expandable device.

In accordance with some embodiments, at least one of the one or more fluid-permeable membrane panels has a first section and a second section, with the first section having a mechanical strength less than the second section.

In accordance with some embodiments, the expanded device has a filling volume of between about 100 cm³ and about 600 cm³. In accordance with some embodiments, the expanded device comprises an open skeletal structure. The expanded device may comprise a toroid. The expanded device may comprise a pair of intersecting toroids. The expanded device may comprise a spheroid.

In accordance with some embodiments, the filling material comprises discrete granules, each of the discrete granules having a volume of less than about 4 cm³ when substantially fully hydrated.

In accordance with some embodiments, the filling material one of structurally and chemically degrades in a gastric environment with a pH of more than about 7. In accordance with some embodiments, the filling material one of structurally and chemically degrades in a gastric environment with a pH of more than about 8.

In accordance with some embodiments, the filling material swells in the presence of gastric fluids to a volume that is about 100 times greater than a dry volume of the filling material.

In accordance with some embodiments, the filling material swells in the presence of gastric fluids from a dry volume to a substantially fully swollen volume within about 5 minutes.

In accordance with some embodiments, the fluid permeable material and the filling material are constructed and arranged to provide an internal pressure of about or more than about 15 kiloPascals (kPa) within the inner volume upon substantially complete hydration of the filling material.

In accordance with some embodiments, the construct further comprises a radiopacifier.

In accordance with some embodiments, the construct is sized to pass through the human esophagus.

In accordance with another aspect of the present invention, there is provided a method of providing an ingestible construct for inducing weight loss in a patient. The method comprises forming a first fluid-permeable membrane panel into a predetermined pattern, joining a first portion of the first fluid-permeable membrane panel to a second portion of the first fluid-permeable membrane panel to form an inner volume, inserting a quantity of a substantially desiccated fluid absorbing material into the inner volume, and joining a third portion of the first fluid-permeable membrane panel to a fourth portion of the first fluid-permeable membrane panel to form an enclosed volume enclosing the quantity of the substantially desiccated fluid absorbing material therein. At least one of joining the first portion of the first fluid-permeable membrane panel to the second portion of the first fluid-permeable membrane panel comprises joining the first portion of the first fluid-permeable membrane panel to the second portion of the first fluid-permeable membrane panel using a joining material that is structurally stable in gastric fluids at a pH of less than about 7 and structurally unstable in gastric fluids at a pH of greater than about 7, and joining the third portion of the first fluid-permeable membrane panel to the fourth portion of the first fluid-permeable membrane panel comprises joining the third portion of the first fluid-permeable membrane panel to the fourth portion of the first fluid-permeable membrane panel using a joining material that is structurally stable in gastric fluids at a pH of less than about 7 and structurally unstable in gastric fluids at a pH of greater than about 7.

In accordance with some embodiments, the method further comprises inserting the first fluid permeable membrane into a dissolvable capsule.

In accordance with some embodiments, the first fluid-permeable membrane panel comprises a first section of the construct, and the method further comprises joining a second section of the construct to the first section of the construct, the second section of the construct comprising a to second fluid-permeable membrane panel enclosing a substantially non-hydrated fluid absorbing gel.

In accordance with some embodiments, forming the construct comprises forming the construct into a shape which expands into a structure having an open skeletal structure upon hydration of the fluid absorbing material in the first section and the fluid absorbing material in the second section.

In accordance with some embodiments, forming the construct comprises forming the construct into a shape which expands into a structure comprising a toroid upon hydration of the fluid absorbing material in the first section and the fluid absorbing material in the second section.

In accordance with some embodiments, forming the construct comprises forming the construct into a shape which expands into a structure comprising a pair of intersecting toroids upon hydration of the fluid absorbing material in the first section and the fluid absorbing material in the second section.

In accordance with some embodiments, forming the construct comprises forming the construct into a shape which expands into a structure comprising a spheroid upon hydration of the fluid absorbing material in the first section and the fluid absorbing material in the second section.

In accordance with some embodiments, the method further comprises administering the construct to a patient, wherein gastric fluids cause the fluid absorbing material to form a swelled material.

In accordance with some embodiments, the method further comprises releasing the swelled material from the enclosed volume by administering to the patient a degradation formulation which disrupts a structural integrity of the enclosed volume.

In accordance with another aspect of the present invention, there is provided a kit. The kit comprises a construct for inducing weight loss in a patient including a one or more fluid-permeable membrane panels and a filler material enclosed within the one or more fluid-permeable membrane panels, wherein the construct in the kit is configured to be in a first compressed state, and configured, responsive to insertion into a patient's stomach, to expand into a second expanded state, and wherein the construct is adapted to release the filler material responsive to contact with a high pH fluid.

In accordance with some embodiments, the kit further comprises means for delivering the construct into a stomach of a patient.

In accordance with some embodiments, the means comprises one of a flexible tube, guidewire, or catheter.

In accordance with some embodiments, the one of the flexible tube, guidewire, or catheter is releasably attachable to the construct.

In accordance with some embodiments, the means comprises a filament attached to the construct and having one or more indicators which indicate to a person administering the construct that the construct is located within in the stomach.

In accordance with some embodiments, the kit further comprises an ingredient of a degradation formulation which when contacted with the construct in a stomach of a patient structurally degrades the construct, releasing the filler material from the one or more fluid-permeable membrane panels.

In accordance with another aspect of the present invention, there is provided a method of inducing weight loss in a patient. The method comprises providing a construct including a one or more fluid-permeable membrane panels and a filler material enclosed within the one or more fluid-permeable membrane panels, wherein the construct is adapted to enter a stomach of a patient in a first compressed state, and responsive to insertion into the stomach, to expand into a second expanded state, and wherein the construct is adapted to release the filler material from one or more fluid-permeable membrane panels responsive to contact with a high pH fluid. The method further comprises instructing the patient to ingest the construct and instructing the patient to ingest a degradation formulation which upon contact with the construct structurally degrades the construct.

In accordance with some embodiments, responsive to ingestion of the construct, the construct contacts gastric fluids and expands to a filling volume of between about 100 cm³ and about 600 cm³.

In accordance with some embodiments, the method further comprises providing a radiopacifier inside the construct.

In accordance with some embodiments, the method further comprises delivering the construct to the stomach of the patient with one of a flexible tube, guidewire, or catheter.

In accordance with some embodiments, the method further comprises delivering the construct to the stomach of the patient with a filament attached to the construct which provides an indication of when the construct is located in the stomach of the patient.

The above and other features of the invention including various novel details of construction and combinations of parts, and other advantages, will now be more particularly described with reference to the accompanying drawings and pointed out in the claims. It will be understood that the particular methods and devices disclosed are shown by way of illustration and not as a to limitation of the invention. The principles and features of this invention may be employed in various and numerous embodiments without departing from the scope of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is an exploded view of a construct in accordance with an embodiment of the present invention;

FIG. 1B illustrates another construct in accordance with an embodiment of the present invention;

FIG. 2 illustrates another construct in accordance with an embodiment of the present invention;

FIG. 3 illustrates another construct in accordance with an embodiment of the present invention;

FIG. 4 illustrates another construct in accordance with an embodiment of the present invention;

FIG. 5A illustrates a membrane panel used in the fabrication of an embodiment of a construct in accordance with an embodiment of the present invention;

FIG. 5B illustrates the membrane panel of FIG. 7A, formed into a tube-like structure;

FIG. 5C illustrates a plurality of tube-like structures as illustrated in FIG. 7B joined together;

FIG. 5D illustrates an expanded construct formed from the plurality of tube-like structures of FIG. 7C;

FIG. 6A illustrates the deployment of an embodiment of a construct;

FIG. 6B illustrates the residence of an embodiment of a construct;

FIG. 6C illustrates the degradation of an embodiment of a construct;

FIG. 6D illustrates the elimination of an embodiment of a construct;

FIG. 7 illustrates another construct in accordance with an embodiment of the present invention;

FIG. 8A illustrates a membrane panel used in the fabrication of an embodiment of a to construct in accordance with an embodiment of the present invention;

FIG. 8B illustrates the membrane panel of FIG. 8A, formed into a tube-like structure;

FIG. 8C illustrates the tube-like structures as illustrated in FIG. 8B divided into separate sections;

FIG. 8D illustrates a toroid formed from the of tube-like structure of FIG. 8C;

FIG. 8E illustrates a construct formed from two toroids as illustrated in FIG. 8D; and

FIG. 9 is a flowchart of an embodiment of a method of fabricating a construct in accordance with an embodiment of the present invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

The invention, in one embodiment, is a bio-compatible construct designed to occupy a portion of the volume of a mammalian stomach and induce a feeling of satiation. In other embodiments, the invention comprises the biocompatible construct and a formulation that, in liquid contact with the construct, causes the construct to degrade into pieces that are small enough to pass out of the stomach through the mammal's digestive tract. In yet other embodiments, the invention is a system comprising the aforementioned construct and formulation and another apparatus that is an aid in deploying the construct in the mammalian stomach.

Aspects and embodiments of the present invention are designed to fill at least a portion of the volume of a mammalian stomach, such as a human stomach. Aspects and embodiments of the present invention are designed to create a sensation of satiation when present in the stomach. It has been discovered that human satiation is triggered not by the true volume of an item in the stomach but by its “filling volume,” defined roughly as the space occupied by the “bounding” figure corresponding to that item. The bounding figure is the figure that would be created when the actual item is enclosed within a deflated balloon (or, in mathematical terms, the surface of minimum potential energy when an idealized elastic film is wrapped around the item). Due to the subjective and highly variable nature of human sensations and due to the different sizes of human stomachs in different people, constructs having different filling volumes may be utilized for different patients. As used herein the term “patient” may encompass both human patients and animals.

In some embodiments the bio-compatible construct is assembled in a desiccated state and subsequently deployed into the stomach where it is exposed to gastric fluids. In some embodiments the construct is deployed through the esophagus, either by swallowing or with a mechanical placement aid. It will be understood that the construct, in its desiccated state, is in some embodiments small enough to pass easily through the esophagus of, for example, an average adult male human. To facilitate ease of passage through the esophagus, the construct, in its desiccated state, may be sized to fit within a ‘000’ capsule. In some embodiments, the construct is designed to expand when exposed to an appropriate fluid, such as gastric fluids in a human stomach, such that it increases in filling volume by a factor of between about 10 times and about 1,000 times.

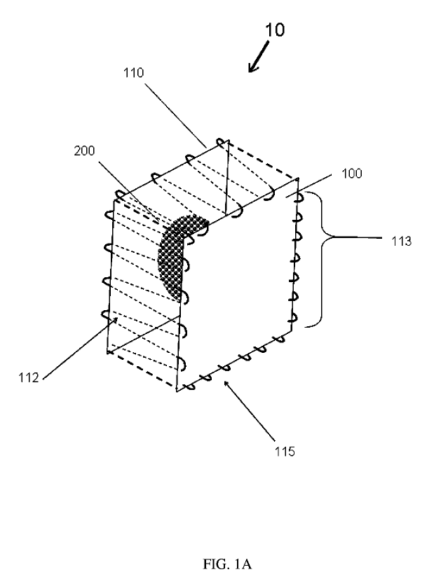

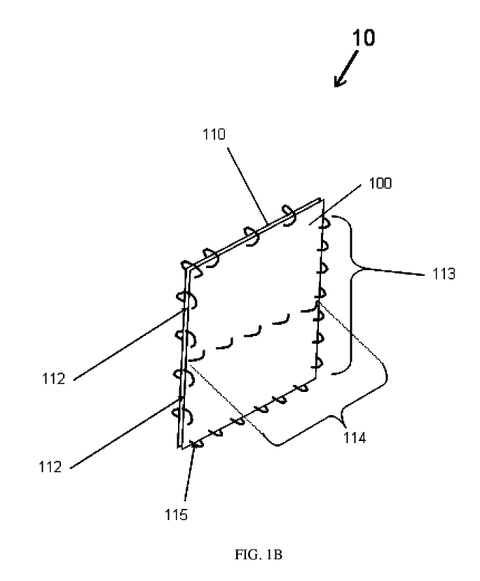

As illustrated in exploded view in FIG. 1A, one embodiment of the invention is a gastric construct 10 comprised of one or more membrane panels 100, a membrane being understood as a thin, sheet-like material in which the thickness of the material is much less than its lateral extent. As used herein, the term “membrane” includes a sheet-like material having multiple layers, a folded sheet-like material, or a sheet-like material having one or more portions thereof joined together. In use, the construct 10 is disposed inside a mammalian stomach, for example, a human stomach, which is at least partially filled with gastric fluids. In FIG. 1A, two nominally identically shaped membrane panels 100 are used to create the construct. The panels 100 are joined at their edges 110 to form one or more enclosed volumes or compartments 112 capable of containing and retaining a filling material 200. In some embodiments the edges 110 are joined by a stitching process while in other embodiments the edges are joined by methods such as glue bonding or ultrasonic welding. In some embodiments one or more edge stitches 113 are sewn with a degradable filament 115.

In some embodiments, as shown in FIG. 1B the one or more panels 100 are also joined away from their edges to form a connected series of contiguous, enclosed volumes 112. The series of contiguous, enclosed volumes 112 are separated by stitching 114. In other embodiments, the series of contiguous, enclosed volumes 112 are separated by other mechanisms, for example, gluing or ultrasonic bonding. A quilt-like construct may be formed by joining multiple individual constructs 10.

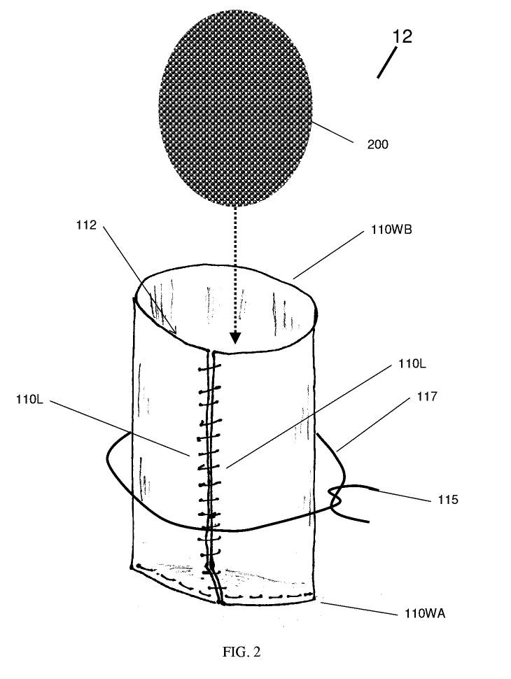

FIG. 2 illustrates a partly fabricated tubular construct 12. A single membrane panel, having an elongated rectangular shape defined by a length L and a width W, where L is greater than W, and where both L and W are much greater than the thickness of the membrane, is shaped into a tube structure by joining its two long edges 110L to each other. As will be understood, a membrane tube, generally, is not self-supporting and generally appears as a collapsed tube. The tube is converted into an open compartment 112 by further drawing together and joining to itself the rim of the tube formed by one of the short edges, edge 110WA. Filling material 200 is disposed inside the thusly formed compartment. The compartment is closed to contain the filling material 200 by drawing together and joining to itself the second rim of the tube, the rim formed by the second short edge 110WB. In FIG. 2 compartment 112 has not yet been closed. In some embodiments, one or more tubular constructs 12 are used to form a series of linked tubes, either by joining a number of individual constructs end-to-end or by partitioning a single tubular construct into a series of sausage-like links by gathering the fabric at mid-points along the tube and closing off the gathered material, for example, by tying it with a loop 117 of filament 115. It will be understood that the sequence of joining edges to form an open compartment, filling compartments, closing compartments, and joining multiple constructs as described herein is by example only and that other sequences that produce equivalent results are within the scope and intent of this invention. Further, in other embodiments, the edges 110WB may be the long edges of the membrane panel 100 and edges 110L may be the short edges.

It will be appreciated that almost any shape construct can be fabricated out of one or more membrane panels that can be joined together along their edges. It will be further appreciated that a construct that comprises one or more enclosed volumes for retaining a filling material can be used to make a three-dimensional, volume-occupying construct, also having almost any shape.

In some embodiments, the construct has a generally open form; that is, a form that allows food to easily pass through the construct instead of having to go around the construct. An open form is less likely to interfere with the normal digestive function of the stomach.

In some embodiments, the construct has a generally skeletal form; that is, a form whose filling volume is spatially defined by several relatively thin ribs, tubes, or bars, rather than a form with a substantially continuous exterior surface. Constructs having such a skeletal form may be described herein as having an “open skeletal structure.” A construct with skeletal form (or “open skeletal structure”) requires less actual volume of material to create the construct as compared to a construct having a similar filling volume, but a monolithic (solid, non-open) structure. The skeletal structure also reduces discomfort associated with having a substantial undigestable “lump” in the stomach as may be experienced with monolithic construct shapes.

One embodiment of a construct comprises a substantially spherical skeletal construct (SSC) 15, as shown in FIG. 3. More specifically, SSC 15 comprises two, intersecting, generally identical and generally toroidal rings 120 forming four ribs 125. Each rib 125 can be considered a line of longitude on a sphere. As illustrated, when the rings 120 are nominally orthogonal, the filling volume of SSC 15 approximates the volume of the surface of revolution of one of the rings (that is, the filling volume approximates the volume of a sphere). Furthermore, SSC 15 is a substantially open structure (an open skeletal structure) which allows stomach contents (for example, food and gastric fluid) to interact and pass through the stomach without significant interference. It will be appreciated that there are many equivalent membrane panel designs that will yield substantially identical constructs. Embodiments of the present invention are not limited to any particular membrane panel design.

Another embodiment of the invention comprises the spherical skeletal construct 15 of FIG. 3 made more rigid by the addition of an equatorial toroidal ring 120A, as illustrated in FIG. 4. The equatorial toroidal ring 120A helps keep the longitudinal ribs 125 separated by the nominal 90 degrees, allowing the ribs 125 to be made less rigid as compared to the ribs 125 in an SSC without an equatorial toroidal ring. In some embodiments, individual ribs 125 of a construct 17 such as illustrated in FIG. 4 are thinner and/or contain a lesser volume of swollen filling material 200 than the ribs 125 of a construct such as illustrated in FIG. 3.

In some embodiments, the membrane panels 100 are fabricated from fabrics made by weaving, knitting, or felting fibers, wherein the fibers are selected from materials known to be biocompatible and stable in a typical human gastric environment, typically an aqueous, acidic environment. Examples of such material include nylon, silk, poly(meth)acrylates, acrylates copolymers, acrylics based co- and terpolymers, poly(vinyl alcohol), poly(ethylene oxide), poly(ethylene), poly(propylene), poly(ethylene terephthalate), poly(tetrafluoroethylene), poly(acrylamide), poly(acrylic acid), polyester, or copolymers of these materials.

In other embodiments, the membrane panels 100 are fabricated from fibers wherein the fibers are selected not only to be stable in a typical human gastric environment but also to be degradable when exposed to an alkaline environment. Examples of these materials include poly(meth)acrylates, acrylates copolymers, acrylics based co- and terpolymers, and poly(acrylic acid).

Additionally, in some embodiments, the membrane panels 100 are permeable to fluids found in the normal gastric environment. This allows these fluids to reach filling material 200 which is enclosed within one or more enclosed volumes formed from the membrane panels of embodiments of the construct. In some embodiments the membrane panels 100 may be a mesh, in which distinct holes are present that allow fluids to pass. The mesh is designed to pass fluids while retaining filling material 200 in both its desiccated and moistened state. Thus, the tightness of the weave or pore size of a mesh is designed in light of the material selected as the filling material 200, for example, the physical size of desiccated and/or swelled granules of the filling material 200.

In some embodiments, the membrane panels 100 are designed to pass through the digestive system without risk of causing an intestinal obstruction. For example, in some embodiments, the panels 100 are no larger than about 100 mm in any dimension, and in some embodiments, no larger than about 50 mm or less in any dimension.

The filling material 200 is in some embodiments a bio-compatible and moisture absorbing material that is selected for its ability to swell to many times its dry volume when exposed to moisture. The filling material 200, in some embodiments, swells by a volumetric factor of between about 10 and about 1,000 responsive to exposure to a fluid, for example, water or gastric fluids. In some embodiments, the filling material 200 swells by a volumetric factor of between about 50 and about 250 responsive to exposure to a fluid, for example, water or gastric fluids. In some embodiments, the filling material 200 swells by a volumetric factor of about 100 responsive to exposure to a fluid, for example, water or gastric fluids. Additionally, the filling material 200 is in some embodiments at least quasi-stable in the gastric environment. Quasi-stability, in this context, means that, in the stomach, the filling material 200 does not degrade to the point of being able to pass through the panels 100 or otherwise escape from enclosed volumes 112 during a pre-determined deployment period that is between about one week and about six months. In some embodiments, the filling material 200 does not degrade to the point of being able to pass through the panels 100 or otherwise escape from enclosed volumes 112 for approximately two months. In some embodiments, the filling material 200 exists as a powder or small granules when dry and as an aggregation of soft particles or a slurry when moistened.

Examples of swellable filling materials for use in various embodiments of the construct include, but are not limited to poly(ethylene glycol), poly(acrylamide), 5 poly(acrylicacid), poly(vinyl alcohol), poly(ethylene oxide), poly(ethyloxazoline), poly(hydroxyethylmethacrylate), proteins, polysaccharides, or copolymers of these materials, or any hydrogel-based material. Polysaccharides can include starch, sodium starch glycolate, cellulose, carboxymethyl cellulose, hydroxyethyl cellulose, hydroxypropyl cellulose, carageenan, chitosan, modified chitosan, chitosan-glycol, hyaluronic acid, chondroitin sulfate, and alginates. Mixtures of materials may also be used. In some embodiments, the swellable filling material will become substantially fully hydrated, for example, absorbing about 75% of its moisture absorbing capacity, within about 5 minutes of contact with a fluid such as water or gastric fluids. As used herein, the term “hydrated” is not limited to describing a state of a material after absorption of water, but may also refer to a state of a material having absorbed other fluids instead of or in addition to water, for example, stomach acid.

The particle size of the filling material 200 is in some embodiments selected to reduce the likelihood that the material will cause obstruction of the bowel once the material is released from the construct. In various embodiments, the swellable particles are generally round and have a diameter range of from about 0.1 mm to about 1.5 mm in their desiccated state, and in some embodiments a diameter of about 1 mm in their desiccated state. In some embodiments, wherein filling material 200 swells by a volumetric factor of about 100, the typical 1 mm particle swells to an approximate diameter of 4.6 mm.

In some embodiments, the volume of desiccated filling material contained in volume(s) 112 is determined based on the selected material's calculated, fully wetted, swollen volume. In some embodiments, the filling material is used to “inflate” the construct to achieve structural rigidity in the deployed state. In some embodiments, upon swelling, the filling material rigidifies the construct sufficiently to prevent stomach contractions from significantly deforming or collapsing the construct, which deformation would reduce the effectiveness of the construct, or from forcing the construct through the pyloric sphincter prior to the intended time of degradation and potentially forming an internal obstruction. In some embodiments, the swelling of the filling material results in a pressure of greater than about 15 kiloPascals (kPa) in the interior of the expanded construct.

In some embodiments the construct is fabricated from one or more individual panels of non-degradable membrane, joined together by stitching. In some embodiments, the membrane is quasi-stable in the gastric environment. The stitches 113 are, in some embodiments, formed using a degradable, bio-compatible filament 115. In some embodiments, the filament 115 is stable or quasi-stable in the normal human gastric environment, which is generally acidic with a pH less than 7, even after the ingestion of a meal (See Dressman et al., Upper Gastrointestinal (GI) pH in Young, Healthy Men and Women, Pharmaceutical Research, Vol. 7, No. 7, 756-61 (1990)). Quasi-stable, to in this context, means retaining a substantial portion of mechanical strength (for example greater than about 20%) for between about a week and about six months, for example, about two months.

In some embodiments of the invention, the construct is designed to remain stable in the stomach until a degradation formulation is introduced. Responsive to introduction of the degradation formulation, the construct degrades into its constituent elements (primarily membrane panels 100 and filling material 200) whereby each constituent element can be safely eliminated from the body by normal intestinal elimination processes. In some embodiments, the construct degrades by a de-joining of membrane panels 100, thereby allowing filling material 200 to escape. In such embodiments, the joining mechanism, for example, stitching or glue, holding the membrane panels together degrades, dissolves, weakens, or releases when subjected to a pre-determined application of an appropriate degradation formulation, which, for example, raises a pH of the fluid in the stomach in which the construct has been placed. In other embodiments, the membrane material itself degrades to release filling material 200. In some embodiments, the membrane material degrades responsive to contact with a degradation formulation that, for example, raises a pH of the fluid in the stomach in which the construct has been placed.

In some embodiments the degradation formulation is introduced orally and at least partially in a liquid format into the stomach, wherein the construct is disposed. In the stomach, the degradation formulation mixes with the resident gastric fluid to become an immersing fluid that substantially bathes the construct. Alternatively, the degradation formulation may be introduced into the stomach in a solid state, as in a tablet or capsule, in some embodiments accompanied by a liquid, whereby the solid is dissolved and becomes the immersing fluid, particularly when mixed with existing gastric fluids. In some embodiments, the degradation formulation includes a base and/or a proton pump inhibitor and/or a histamine blocker designed to raise the pH of the stomach above about 7. Studies have illustrated that responsive to the administration of 150 mg of ranitidine supplemented with 20 ml of an 8.4% sodium bi-carbonate solution subjects experienced an increase in gastric pH to greater than 7, while most subjects' gastric pH rose to greater than or equal to 9 for an extended period of time, typically greater than 2 hours. (See, e.g., E. M. Thompson et al., Combined treatment with ranitidine and saline antacids prior to obstetric anesthesia, Anesthesia, Vol. 39, 1086-90 (1984); E. M. Thompson et al., Combined treatment with ranitidine and sodium bicarbonate prior to obstetric anesthesia, Anesthesia, Vol. 41, 1202-06 (1986); H. M. L. Mathews et al., Sodium bicarbonate as a single dose antacid in obstetric anesthesia, Anesthesia, Vol. 44, 590-91 (1989).)

The membrane and/or the joining material (for example, the filament 115) may be composed of any biocompatable polymer or natural material known to those in the art. In some embodiments, the material can include, but not be limited to, the following materials: poly(meth)acrylates, acrylates copolymers, acrylics based co- and terpolymers, poly(dioxanone), poly(glycolic acid), poly(lacticacid), poly(vinyl alcohol), poly(ethylene oxide), poly(caprolactone), alginate, polysaccharides, or co-polymers thereof.

In some embodiments, the individual constituent elements of the construct may degrade into smaller and/or more elimination-suitable parts after the administration of the degradation formulation. For example, the individual membrane panels 100 may degrade into smaller “scraps” of membrane when exposed to a high pH gastric environment. As another example, a gelatinous mass of filling material 200 may degrade into smaller masses or devolve into a softer consistency in the high pH gastric environment.

In some embodiments, the construct is deployed to the stomach through the esophagus. In one embodiment the construct includes an ingestible, dissolvable capsule. In some embodiments, the membrane enclosing the filling material may be rolled or wadded to fit into a capsule, such as a ‘000’ gelatin capsule, having a length of approximately 26 mm, a diameter of approximately 9.9 mm, and a volume of approximately 1.4 ml, which capsule is known to be swallowable by a substantial percentage of adult human subjects. Gelatin capsules rapidly dissolve in the stomach, releasing their contents, in this case, the construct. The filling material is then able to absorb moisture from the stomach fluids, swelling the filling material and thereby expanding the membrane to its design size and shape. In some embodiments the capsule dissolves and the filling material swells to substantially its final swelled volume (for example, 75% or more of its final volume) in less than about 30 minutes, and in some embodiments, in less than about 5 minutes after reaching the stomach.

In some embodiments the capsule may be attached to a distal end of a tethering filament. When the capsule is ingested by an individual, the proximal end of the tethering filament is secured outside of his or her mouth. In some embodiments, the length of the tethering filament is calibrated to match the distance between the particular individual's mouth and stomach so that the capsule is automatically positioned within the stomach and cannot immediately pass through the pylorus. In other embodiments, the tethering filament is marked with an indicator which is used to determine when the capsule has entered into a patient's stomach. Once the capsule has dissolved and released the construct into the stomach the tethering filament may be retrieved through the mouth.

Alternatively, in some embodiments the capsule may be attached to the distal end of a commercially available capsule delivery system, such as the AdvanCE® capsule delivery device from US Endoscopy, Mentor, Ohio. This device allows for direct placement of large capsules such as video endoscopy capsules into the stomach of individuals with oropharyngeal or mechanical dysphagia, gastroparesis and known or suspected anatomical abnormalities who cannot swallow these capsules. Other delivery devices may comprise a flexible tube with sufficient rigidity and length to advance through the esophagus and place the capsule into the patient's stomach. In some embodiments, the flexible tube may comprise, but not be limited to, a catheter, guidewire, plastic tube, or any pre-existing endoscopic capsule delivery device. The flexible tube may be affixed to the construct using an adhesive, suction, or a tethering wire. The construct and the tube may be guided through the mouth into the oropharynx, down the esophagus, past the lower esophageal sphincter, and into the gastric chamber. The flexible tube can be designed to have a length that approximates the distance between the oropharynx and the pylorus (typically 50-80 cm) to facilitate proper positioning of the construct in the stomach. If the capsule or tablet accidentally passes through the pyloric sphincter into the duodenum, the tube can be used to pull the construct back into the stomach. Once the construct is appropriately positioned in the stomach, the tube is detached from the swellable construct using methods including, but not limited to: a bolus of air, a bolus of a liquid, the degradation of a supporting wire, the retraction of a supporting wire, and/or the degradation of an adhesive. To facilitate passage of the tube through the oropharynx, the patient's throat can be temporarily anesthetized using an anesthetic spray (e.g., xylocalne spray).

In accordance with an embodiment of a safety feature, the membrane may be designed to burst if the pressure in the stomach reaches a critical level or if the construct migrates into the duodenum or expands in the esophagus or duodenum. In this fashion, if a patient either ingests more than a desired number of constructs, or overeats following the ingestion of a desired number of constructs, or if the construct either expands in the esophagus or duodenum or migrates into the duodenum or small intestine, the expanded construct will burst and shrink before the patient is harmed or an intervention is required. The burst pressure may be designed to be well below the burst (perforation) pressure of the stomach, esophagus, or duodenum. In some embodiments, the membrane may be fabricated to have a structurally weak section with a known burst strength. For example, one or more lines of perforations may be formed in a section of the membrane that may tear upon application of more than a desired pressure to the construct.

In another aspect the invention comprises a kit, or system, for inducing weight loss in a human. In some embodiments, the kit includes one or more of an embodiment of a construct as described herein, one or more ingredients for a degradation formulation, a construct delivery mechanism (for example, an AdvanCE® capsule delivery device and/or tethering filament), and instructions for administering the construct to a patient and/or instructions to be followed by a patient receiving the construct. In addition, the kit may include a container for the contents of the kit.

An embodiment of a swelled construct is illustrated in FIG. 3. The construct includes two nominally orthogonal, intersecting, generally toroidal, structures composed of multiple, linked, approximately cylindrical compartments whose membrane panels comprise flexible fabric panels. In some embodiments, the panels are stitched together with a filament to enclose a filling material comprising a fluid-absorbing gel.

In some embodiments, the fabric (the membrane) is a biocompatible, synthetic polymer such as polyester knit mesh 0.12 mm in thickness and pore size of 0.05 mm, available, for example, from Biomedical Structures of Warwick, R.I. In other embodiments the fabric may include other materials such as polypropylene knit mesh 0.36 mm in thickness and with pore size of 0.05 mm or poly-tetra-fluoro-ethylene (PTFE) knit mesh 0.38 mm in thickness and with pore size of 0.05 mm, both of which are available, for example, from Biomedical Structures of Warwick, R.I. Embodiments of the present invention are not limited to membranes having any particular thickness or pore size unless explicitly set forth in the claims.

In some embodiments, the filament is a biocompatable polymer, co-polymer or natural material that remains structurally stable (for example, having mechanical properties such as strength which do not change over time) or substantially structurally stable and/or unmodified at pH 1 through pH 7 but degrades (for example, by dissolving) at a pH greater than about 7. In other embodiments the pH at which the filament becomes structurally unstable is greater than about 8. In some embodiments the filament comprises poly(methacrylic acid-co-methyl methacrylate), available as EUDRAGIT S-100 from Evonik Industries of Darmstadt, Germany. In other embodiments the filament comprises poly(methyl acrylate-co-methyl methacrylate-co-methacrylic acid) co-polymer, available as EUDRAGIT FS-30D from Evonik Industries of Darmstadt, Germany. In some embodiments, the filament has an outer diameter of 0.016 inches and a tensile strength of at least 20 Newtons, although embodiments of the present invention are not limited to including filaments of any particular diameter or tensile strength. In some embodiments, the to filament has no intrinsic dissolution rate at a pH of less than about 7.0, and an intrinsic dissolution rate of about 50 mg/(g*min) at a pH of about 7.0 and about 250 mg/(g*min) at a pH of about 8.0. In other embodiments the filament may exhibit different dissolution rates. The dissolution rate of the filament may be tailored by choosing a desired composition of the filament.

The filling material is, in some embodiments, a fluid-absorbing gel comprising a biocompatible material that can swell in the stomach, such as chitosan. In some embodiments, the chitosan is covalently coupled with a vinyl group-containing substrate such as glycidyl methacrylate to form a vinyl-group-containing chitosan. A superporous hydrogel is then formed in the presence of a radioopaque marker that is impregnated within the hydrogel by mixing together at equilibrium in deionized water:

10 g of the vinyl-group-containing chitosan,

5 g barium sulfate

0.9 mL 50% weight/volume acrylamide and 0.5% methylene bisacrylamide,

0.6 5 mL of 50% weight/volume sulfopropylacrylate,

0.1 mL 10% weight/volume pluronic F127 (surfactant),

0.03 mL 50% acrylic acid,

0.04 mL 20% volume/volume tetra-methyl-ethylene-diamine, and

100 mg Ac-Di-Sol.

wherein the above are available, for example, from Sigma-Aldrich of St. Louis, Mo.

To initiate the polymerization process, 0.06 mL of 20% volume/volume ammonium persulfate (available from Sigma-Aldrich of St. Louis, Mo.) is then added and allowed to mix for 2 minutes. 60 mg of sodium bicarbonate (available from Sigma-Aldrich of St. Louis, Mo.) is then mixed into the solution, and it is allowed to foam. The resulting product is ground using an analytic mill (for example, an All mill available from IKA of North Carolina, United States) into granules with diameter of about 1.0 mm

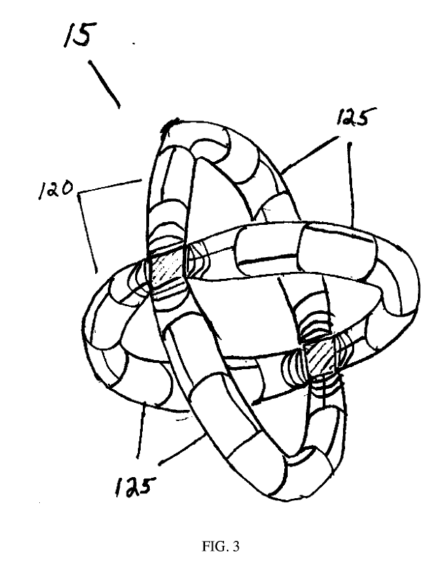

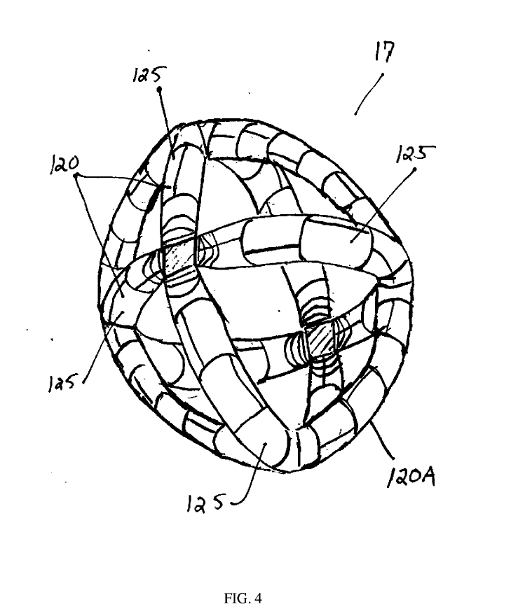

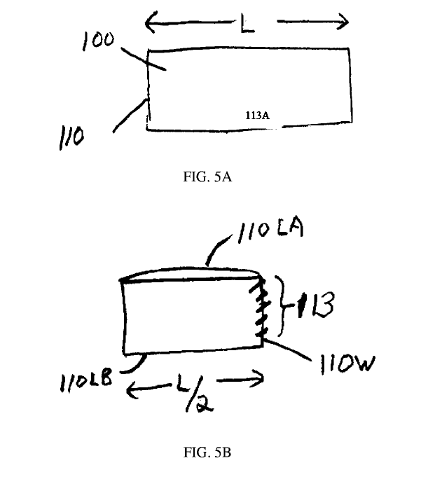

In some embodiments, a construct comprising a pair of joined toroids has a final filling volume of about 400 cm³ when expanded in a patient's stomach. In some embodiments, to achieve a final filling volume of 400 cm³, each toroid is constructed from about 14 tubular sections, each created from a membrane panel 100 6 cm long×2 cm high as shown in FIG. 5A. Each panel is folded in half to form a rectangle 3 cm long×2 cm high and joined with stitches 113 along its 2 cm heightwise open edge with filament to form a 2 cm high, collapsed tube, with the top and bottom ends of each tube open, as illustrated in FIG. 5B. A pre-defined amount of filling material is to deposited inside each collapsed tube. Using a fluid-absorbing gel which swells to about 30 times its initial volume responsive to exposure to fluid, the construct contains a total of about 4 mL of desiccated fluid-absorbing gel to achieve a final filling volume of 400 cm³. Each toroid contains about 2 mL of desiccated fluid-absorbing gel and therefore each tubular section will be filled with about 0.14 mL (2 mL/14) or gram equivalent of fluid-absorbing gel. The tubular sections are positioned end-to-end with approximately 0.1 cm of overlapping material. The ends of each tubular section are joined with stitches 113A (for clarity indicators are not provided for each section of stitching 113, 113A) in the region of overlapping material using alkaline degradable filament, which stitching both closes the ends of both adjacent tubular sections, so the filling material cannot migrate between sections, while simultaneously attaching the adjacent tubular sections to each other. The stitching 113 of each individual tubular section need not be aligned with the stitching 113 of other tubular sections. As shown in FIG. 5C, this creates a chain of collapsed tubular compartments 28 cm in length with two open ends 110LA, 110LB. The chain of compartments is then folded back on itself to position the open ends with about 0.1 cm of overlapping material. The alkaline degradable filament is used to stitch the final tubular sections closed and to join them to form a collapsed toroid with an outer circumference of about 28 cm. A second toroid is formed in a similar fashion and then stitched to the first toroid so that they are orthogonal to one another as illustrated in FIG. 5D, to create the desired spherical skeletal construct 15, where the collapsed toroids are shown in their swollen, expanded state for clarity.

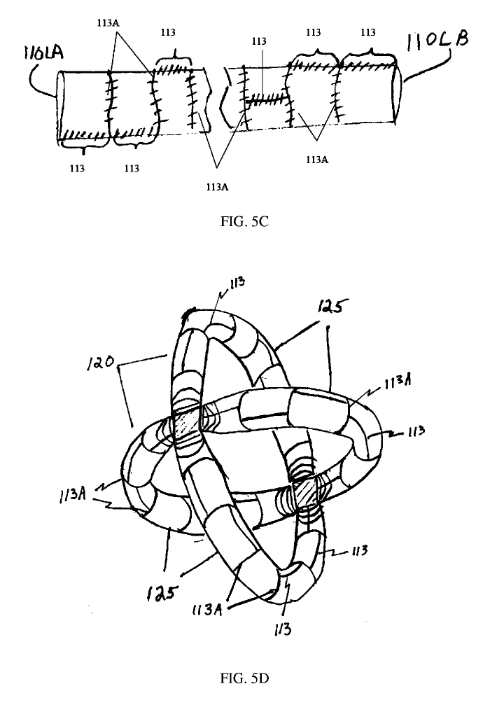

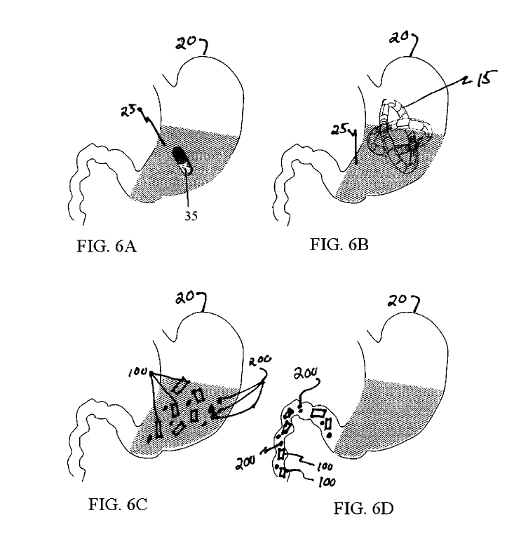

Once assembled, the construct is compressed and placed inside a 000 capsule 35 (available from Capsugel from Greenwood, S.C.) that remains stable and unmodified (not dissolved) in the esophagus but rapidly disintegrates in the stomach. As shown in FIG. 6A-FIG. 6D, the capsule 35 is swallowed and enters the stomach 20 (FIG. 6A) where the capsule 35 dissolves and releases the construct 15. Water in the stomach and/or gastric fluids 25 diffuse across the membrane and are absorbed into the fluid-absorbing gel. The fluid-absorbing gel swells and causes the construct 15 to swell to its final shape (FIG. 6B). In some embodiments the construct remains stable and substantially unmodified in shape and composition for about 2 months or until the pH of the stomach exceeds about 7, or in some embodiments until the pH exceeds about 7.5 or about 8.

Construct degradation can be is accomplished by the ingestion of a degradation formulation that elevates the gastric pH beyond 7, or in some embodiments, beyond 8. In some embodiments, the degradation formulation includes about 20 mL of an 8.4% solution of sodium bicarbonate taken at least 2 hours after ingestion of 150 mg of ranitidine, which has been demonstrated to raise the to stomach pH to greater than 8. As has been described, the elevated gastric fluid pH dissolves (e.g., degrades) the filament.

With the filament stitching at least partially degraded, the fluid-absorbing gel 200 spills out and forms a slurry in the stomach that can pass through the pylorus into the small intestines and be excreted (FIG. 6C). The cylindrical compartments of membrane material unfold after the filament dissolves to form 6 cm×2 cm rectangles 100 that can pass through the pylorus into the small intestines and be excreted (FIG. 6D). For some embodiments deployed in a human stomach, the process of total degradation (that is, the devolution of the construct into excretable elements) is completed in approximately 30 minutes. The exact time that may be required for degradation of the construct is dependent on the specific condition of the stomach.

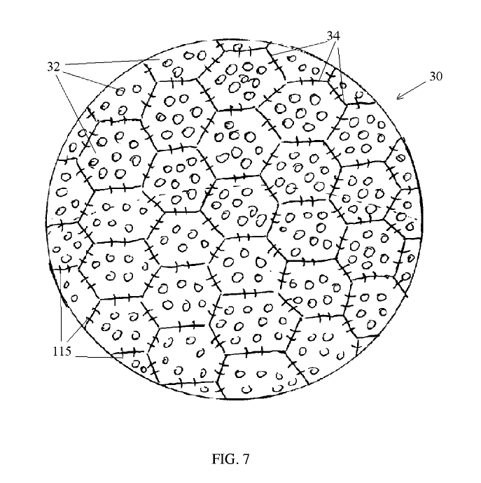

Another embodiment of the construct is illustrated in FIG. 7. In this embodiment the expanded construct 30 comprises a sphere composed of regular hexagonal panels 32 of fabric which can be stitched together with a filament 115 to enclose a fluid-absorbing gel. A final filling volume of about 400 cm³ and surface area of about 262.4 cm² can be achieved using 25 regular hexagonal panels with side lengths 34 of about 2 cm stitched together with filament 115. In other embodiments, the expanded construct 30 may form an imperfect sphere, for example, a slightly flattened sphere or an egg-shaped structure. Both perfect and imperfect spherical structures may be referred to herein using the term “sphereoid.”

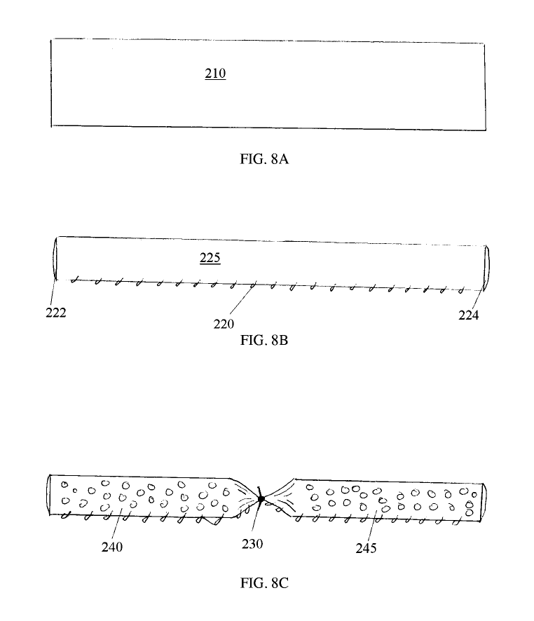

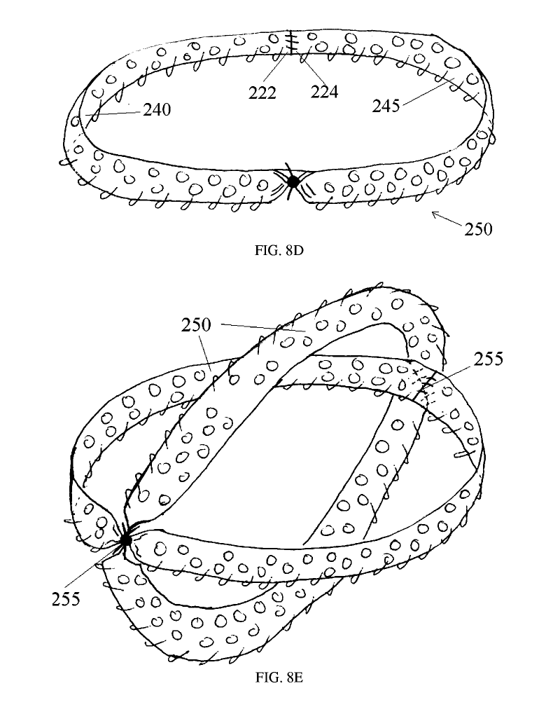

In yet another embodiment illustrated in FIG. 8, the construct is made with two toroids 250 (FIG. 8D and FIG. 8E) each constructed from two large sheets of membrane 210 (FIG. 8A) made from material that remains stable in the stomach until it is alkalinized by a degradation formulation. In some embodiments the membrane comprises poly(methacrylic acid-co-methyl methacrylate), available as EUDRAGIT S-100 from Evonik Industries of Darmstadt, Germany. The final filling volume is 400 cm³. To achieve a final filling volume of 400 cm³, each toroid is typically constructed from a sheet of membrane 210 28 cm long×6 cm high. The sheet of membrane 210 is folded in half to form a rectangle 28 cm long×3 cm high and stitched along its 28 cm lengthwise open edge with filament 220 to form a collapsed tube 225 (FIG. 8B), leaving the ends of the tube 222, 224 open. A knot 230 is then tied that divides the collapsed tube into two tubular sections 240, 245 so that filling material cannot migrate between sections (FIG. 8C). Each tubular section 240, 245 is then filled with 1 mL or gram-equivalent of fluid-absorbing gel. The collapsed tube is then folded back on itself to position the open ends with 0.1 cm of overlapping material. The alkaline degradable filament is used to stitch the collapsed tube closed and to join them to form a toroid 250 with outer circumference 28 cm (FIG. 8C). A second toroid 250 is formed in an identical fashion and then stitched to the first toroid with stitching 255 so that they are orthogonal to one another (FIG. 8E). Upon alkalinization of the stomach with the degradation formulation, both the filament and the fabric dissolve, leaving only the swollen fluid-absorbing gel to pass through the pylorus and be excreted.

In yet another embodiment, the membrane, filament, or both comprise one or more materials that slowly dissolve in the stomach without the need for a degradation formulation. Examples of such materials include oxidized cellulose (for example, Surgicel Nu-Knit Hemostat available from Ethicon), polydioxanone (for example, PDS I available from Ethicon), or poly (lactide-co-glycolide) (for example, Polysorb available from Alkermes of Wilmington, Ohio).

In yet other embodiments, the fluid-absorbing gel is impregnated with a different radioopaque material such as titanium dioxide or bismuth-based compounds that allows visualization of the fluid-absorbing gel on x-ray.

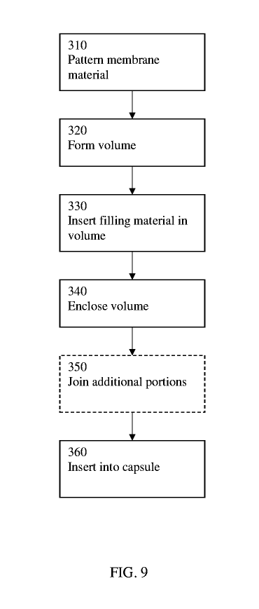

Other aspects and embodiments of the invention are directed to a method of assembling a construct. As illustrated in the flowchart of FIG. 9, embodiments of the method include an act 310 of patterning a membrane material. The membrane material may be patterned into a shape such that when expanded by the swelling of a swellable material, the expanded membrane may take the form of a desired shape, for example a shape having an open skeletal structure such as a toroid or a pair of joined intersecting toroids. Following patterning of the membrane, in act 320, an open volume is formed from the patterned membrane. The open volume may be formed by joining one portion of the membrane to another to form, for example, a pocket, pouch, or tube. In act 330, filling material, such as fluid absorbing material in for example, substantially desiccated granule form, is added to the open volume. An amount of filling material inserted into the open volume may be calculated to provide a sufficient increase in volume after swelling of the filling material to fully expand the volume in which the filling material is enclosed, or to provide a desired level of internal pressure in the volume. In act 340, open portion(s) of the open volume are joined to form an enclosed volume about the filling material. In optional act 350, one or more additional sections of membrane material are joined to the section formed in acts 310-340. These additional sections of membrane material may also include enclosed volumes enclosing quantities of fluid absorbing material. In alternate embodiments, multiple separate sub volumes may be formed from the enclosed volume formed in act 340 by, for example joining additional portions of the membrane material together. In act 360 the membrane(s) including the fluid absorbing material in the enclosed volume(s) is to inserted into a capsule which may later be administered to a patient to introduce the capsule to the patient's stomach. As described above, upon administering the construct to a patient, gastric fluids cause the fluid absorbing material to form a swelled material, expanding the enclosed volume(s) of the membrane. A degradation formulation may later be given to the patient to disrupt the structural integrity of the expanded enclosed volume(s), releasing the swelled material.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A construct for inducing weight loss in a patient comprising:
an expandable device constructed and arranged to enter a stomach of a patient in a first compressed state, and responsive to insertion into the stomach, to expand into a second expanded state, and
wherein a portion of the expandable device is constructed to structurally degrade responsive to contact with a high pH fluid.

2. The construct of claim 1, wherein the second expanded state comprises an open skeletal structure.

3. The construct of claim 1, wherein the expandable device includes:
one or more fluid-permeable membrane panels defining at least part of an inner volume; and
a filling material contained in the inner volume, the filling material including a substance which absorbs moisture and swells in the presence of gastric fluids;
wherein the one or more fluid-permeable membrane panels and the filling material are constructed and arranged such that swelling of the filling material causes the expandable device to expand into the second expanded state; and
wherein the expandable device is constructed and arranged such that in the presence of a high pH fluid an opening forms into the inner volume, the opening having a size sufficient to allow the filling material to escape the inner volume.

4. The construct of claim 3, wherein the expandable device is structurally stable in a gastric environment with a pH of less than about 7.

5. The construct of claim 4, wherein the portion of the expandable device structurally degrades in a gastric environment with a pH greater than about 7.

6. The construct of claim 3, wherein the one or more fluid-permeable membrane panels comprise one of polyester, poly-tetra-fluoro-ethylene, and nylon.

7. The construct of claim 3, wherein the expandable device further includes a joining material coupled to the one or more fluid-permeable membrane panels and constructed to join a first portion of the one or more fluid-permeable membrane panels to one of a second portion of the one or more fluid-permeable membrane panels and a second membrane panel to form the inner volume.

8. The construct of claim 7, wherein the joining material is structurally stable in a gastric environment with a pH of less than about 7.

9. The construct of claim 8, wherein the joining material degrades in a gastric environment to with a pH greater than about 7.

10. The construct of claim 8, wherein the joining material comprises stitching including a filament formed of one of poly(methacrylic acid-co-methyl methacrylate), poly(methyl acrylate-co-methyl methacrylate-co-methacrylic acid) co-polymer, and another methacrylic acid-methyl methacrylate co-polymer.

11. The construct of claim 7, wherein the one or more fluid-permeable membrane panels comprises a plurality of fluid-permeable membrane panels coupled together using the joining material.

12. The construct of claim 11, wherein the one or more fluid-permeable membrane panels each have a maximum dimension of less than about 10 cm.

13. The construct of claim 3, further comprising a dissolvable capsule containing the expandable device.

14. The construct of claim 3, wherein at least one of the one or more fluid-permeable membrane panels has a first section and a second section, with the first section having a mechanical strength less than the second section.

15. The construct of claim 3, wherein the expanded device has a filling volume of between about 100 cm³ and about 600 cm³.

16. The construct of claim 3, wherein the expanded device comprises an open skeletal structure.

17. The construct of claim 16, wherein the expanded device comprises a toroid.

18. The construct of claim 17, wherein the expanded device comprises a pair of intersecting toroids.

19. The construct of claim 3, wherein the expanded device comprises a sphereoid.

20. The construct of claim 3, wherein the filling material comprises discrete granules, each of the discrete granules having a volume of less than about 4 cm³ when substantially fully hydrated.

21. The construct of claim 3, wherein the filling material swells in the presence of gastric fluids to a volume that is about 100 times greater than a dry volume of the filling material.

22. The construct of claim 3, wherein the filling material swells in the presence of gastric fluids from a dry volume to a substantially fully swollen volume within about 5 minutes.

23. The construct of claim 3, further comprising a radiopacifier.

24. The construct of claim 3, wherein the construct is sized to pass through the human esophagus.

25. A method of providing an ingestible construct for inducing weight loss in a patient comprising:
forming a first fluid-permeable membrane panel into a predetermined pattern;
joining a first portion of the first fluid-permeable membrane panel to a second portion of the first fluid-permeable membrane panel to form an inner volume;
inserting a quantity of a substantially desiccated fluid absorbing material into the inner volume; and
joining a third portion of the first fluid-permeable membrane panel to a fourth portion of the first fluid-permeable membrane panel to form an enclosed volume enclosing the quantity of the substantially desiccated fluid absorbing material therein;
wherein at least one of joining the first portion of the first fluid-permeable membrane panel to the second portion of the first fluid-permeable membrane panel comprises joining the first portion of the first fluid-permeable membrane panel to the second portion of the first fluid-permeable membrane panel using a joining material that is structurally stable in gastric fluids at a pH of less than about 7 and structurally unstable in gastric fluids at a pH greater than about 7, and joining the third portion of the first fluid-permeable membrane panel to the fourth portion of the first fluid-permeable membrane panel comprises joining the third portion of the first fluid-permeable membrane panel to the fourth portion of the first fluid-permeable membrane panel using a joining material that is structurally stable in gastric fluids at a pH of less than about 7 and to structurally unstable in gastric fluids at a pH of greater than about 7.

26. The method of claim 25, further comprising inserting the first fluid-permeable membrane panel into a dissolvable capsule.

27. The method of claim 25, wherein the first fluid-permeable membrane panel comprises a first section of the construct, and the method further comprises joining a second section of the construct to the first section of the construct, the second section of the construct comprising a second fluid-permeable membrane panel enclosing a substantially non-hydrated fluid absorbing gel.

28. The method of claim 27, wherein forming the construct comprises forming the construct into a shape which expands into a structure having an open skeletal structure upon hydration of the fluid absorbing material in the first section and the fluid absorbing material in the second section.

29. The method of claim 28, wherein forming the construct comprises forming the construct into a shape which expands into a structure comprising a toroid upon hydration of the fluid absorbing material in the first section and the fluid absorbing material in the second section.

30. The method of claim 28, wherein forming the construct comprises forming the construct into a shape which expands into a structure comprising a pair of intersecting toroids upon hydration of the fluid absorbing material in the first section and the fluid absorbing material in the second section.

31. The method of claim 27, further comprising administering the construct to a patient, wherein gastric fluids cause the fluid absorbing material to form a swelled material.

32. The method of claim 31, further comprising releasing the swelled material from the enclosed volume by administering to the patient a degradation formulation which disrupts a structural integrity of the enclosed volume.

33. A kit comprising:
a construct for inducing weight loss in a patient including:
one or more fluid-permeable membrane panels; and
a filler material enclosed within the one or more fluid-permeable membrane panels;
wherein the construct in the kit is configured to be in a first compressed state, and configured, responsive to insertion into a patient's stomach, to expand into a second expanded state, and
wherein the construct is adapted to release the filler material responsive to contact with a high pH fluid.

34. The kit of claim 33, further comprising means for delivering the construct into a stomach of a patient.

35. The kit of claim 34, wherein the means comprises one of a flexible tube, guidewire, or catheter.

36. The kit of claim 35, wherein the one of the flexible tube, guidewire, or catheter is releasably attachable to the construct.

37. The kit of claim 36, wherein the means comprises a filament attached to the construct and having one or more indicators which indicate to a person administering the construct that the construct is located within the stomach.

38. The kit of claim 33, further comprising an ingredient of a degradation formulation which when contacted with the construct in a stomach of a patient structurally degrades the construct, releasing the filler material from the one or more fluid-permeable membrane panels.

39. A method of inducing weight loss in a patient comprising:
providing a construct including
one or more fluid-permeable membrane panels, and
a filler material enclosed within the one or more fluid-permeable membrane panels,
wherein the construct is adapted to enter a stomach of a patient in a first compressed state, and responsive to insertion into the stomach, to expand into a second expanded state, and
wherein the construct is adapted to release the filler material from the one or more fluid-permeable membrane panels responsive to contact with a high pH fluid;
instructing the patient to ingest the construct; and
instructing the patient to ingest a degradation formulation, which upon contact with the construct structurally degrades the construct.

40. The method of claim 39, wherein, responsive to ingestion of the construct, the construct contacts gastric fluids and expands to a filling volume of between about 100 cm³ and about 600 cm³.

41. The method of claim 40, further comprising providing a radiopacifier inside the construct.

42. The method of claim 40, further comprising delivering the construct to the stomach of the patient with one of a flexible tube, guidewire, or catheter.

43. The method of claim 40, further comprising delivering the construct to the stomach of the patient with a filament attached to the construct which provides an indication of when the construct is located in the stomach of the patient.
